(19) United States
(12) Patent Application Publication
(10) Pub. No.: US 2004/0186567 A1
(43) Pub. Date: Sep. 23, 2004

(54) Method for improving corneal inlays

(76) Inventor: Gary M. Kawesch, Santa Clara, CA (US)

Correspondence Address: Bourque & Associates, P.A., Suite 301, 835 Hanover Street, Manchester, NH, 03104, US

(21) Appl. No.: 10/803,013

(22) Filed: Mar. 16, 2004

Related U.S. Application Data
(60) Provisional application No. 60/455,513, filed on Mar. 18, 2003.

(51) Int. Cl. A61F 2/14

(52) U.S. Cl. 623/5.15; 606/107

(57) ABSTRACT

A method of preventing rotation, de-centering, and/or backwards implantation of a corneal inlay relative to a cornea is disclosed. According to one embodiment, the corneal inlay includes a plurality of microscopic spike like protrusions disposed on the bottom of a corneal inlay that engage the surface the cornea. Alternatively, apertures may be ablated in the cornea that are sized and shaped to accept at least a portion of the corneal inlay such as a protrusion. The apertures and protrusions form a “lock and key” type arrangement that prevents movement of the corneal inlay and aid in the precise placement and maintenance of the placement of the inlay. The corneal inlay and aperture preferably include a non-circular shape.

### CROSS-REFERENCE TO RELATED APPLICATIONS

This application claims the benefit of the U.S. provisional application No. 60/455,513, filed Mar. 18, 2003.

### TECHNICAL FIELD

The present invention relates to corneal inlays and more particularly, to a method for preventing rotation, de-centering, and backward implantation of corneal inlays.

### BACKGROUND

It is known to correct ametropia of an eye by a process involving the ablation of the cornea with a laser beam to vary the shape of the cornea. However, while this method is effective at correcting certain types of ametropia, complications arise if the cornea changes after the surgery. Recently, corneal inlays have been implanted within the stroma in a pocket or under a flap to correct the shortcomings of the laser surgery or to provide an alternative. Should the patient's vision change after the implantation of the corneal inlay, the old inlay is simply removed and a new inlay, having the desired necessary contours, is implanted in its place. Alternatively, the procedure could be reversed by the removal of the inlay.

Traditional corneal inlays suffer from several known problems. For example, traditional corneal inlays are round in shape and often have a complex curvature designed to correct the ametropia of a specific cornea. For instance, one portion of a corneal inlay may be thicker than another portion. Toric and wavefront inlays are not radially symmetric. Consequently, the orientation of the inlay is often of great importance. If the inlay is even a little bit off center or is not properly rotated, the ability of the corneal inlay to correct the ametropia will be severely degraded, or worse, could result in a greater ametropia. Unfortunately, traditional corneal inlays are prone to movement, including rotatation or de-centration. Moreover, due to the flexible nature of traditional corneal inlays as well as the inability to differentiate between the front and back sides of these corneal inlays with the naked eye, it is possible to accidentally implant the corneal inlay inwards out during surgery. This, of course, reduces the ability of the corneal inlay to correct the ametropia and results in a condition similar to the focusing problems associated with the Hubbell Space Telescope when it was first launched.

Accordingly, there exists a need for an improved corneal inlay that prevents rotation of the corneal inlay. There also exists the need for an improved corneal inlay that will not slip or de-center itself. The improved corneal inlay should preferably reduce the likelihood that the corneal inlay will be accidentally implanted “inside-out” (backwards).

### SUMMARY OF THE INVENTION

The present invention features a method of preventing rotation, de-centering, and/or backwards implantation of a corneal inlay relative to a cornea. According to one embodiment, the corneal inlay includes a plurality of microscopic spikes disposed on a bottom surface of the corneal inlay. The spikes engage the surface of the bed of the cornea under the flap much like “cleats”, thus preventing movement of the corneal inlay. Alternatively, apertures may be ablated in the cornea using an excimer laser. The apertures may be sized and shaped to accept at least a portion of the corneal inlay. Alternatively, or in addition to, the apertures may be sized and shaped to accept protrusions disposed on the bottom surface of the corneal inlay. The apertures and protrusions form a “lock and key” or “peg-in-hole” type arrangement that prevents movement of the corneal inlay and aid in the precise placement of the inlay. The corneal inlay may include round or non-round shapes and may also include indicia to identify the top and bottom surface and to aid in the orientation of the corneal inlay.

### DESCRIPTION OF THE DRAWINGS

These and other features and advantages of the present invention will be better understood by reading the following detailed description, taken together with the drawings wherein:

FIG. 1 is top partial view of a cornea and the corneal inlay according to one embodiment of the present invention;

FIG. 2A is a top view of the corneal inlay as shown in FIG. 1 having a plurality of spikes according to one feature of the present invention;

FIG. 2B is a cross sectional view of the embodiment shown in FIG. 2A along the line 2B;

FIG. 3A is a partial side view of one embodiment of a cornea having an aperture according to another aspect of the present invention;

FIG. 3B is a cross sectional view of the cornea shown in FIG. 3A along the line 3B;

FIGS. 3C is a partial side view of one embodiment of a cornea having two apertures according to another aspect of the present invention;

FIG. 3D is a cross sectional view of the cornea shown in FIG. 3C along the line 3D;

FIG. 3E is a bottom view of the corneal inlay as shown in FIG. 1 having a plurality of protrusions wherein the protrusions include a partial arc;

FIG. 3F is a cross sectional view of the corneal inlay shown in FIG. 3E along the line 3F;

FIG. 3G is a bottom view of the corneal inlay as shown in FIG. 1 having a single of protrusion wherein the protrusion is in the shape of an arc;

FIG. 3H is a cross sectional view of the corneal inlay shown in FIG. 3G along the line 3H;

FIG. 3I is a bottom view of the corneal inlay as shown in FIG. 1 having a plurality of protrusions wherein the protrusions are generally straight segments;

FIG. 3J is a cross sectional view of the corneal inlay shown in FIG. 3I along the line 3J;

FIGS. 4A-4E are top views of various shapes of corneal inlay shown in FIG. 1 according to one aspect of the present invention;

FIG. 5A is a bottom view of the corneal inlay as shown in FIG. 1 having a protrusion and two apertures;

FIG. 5B is a cross sectional view of the corneal inlay shown in FIG. 5A along the line 5B;

FIG. 6A is a bottom view of the corneal inlay as shown in FIG. 1 having a protrusion wherein the protrusion and the corneal inlay have substantially the same diameter;

FIG. 6B is a cross sectional view of the corneal inlay shown in FIG. 6A along the line 6B;

FIG. 7A is a bottom view of the corneal inlay as shown in FIG. 1 having a single protrusion;

FIG. 7B is a cross sectional view of the corneal inlay shown in FIG. 7A along the line 7B;

FIG. 8A is a bottom view of the corneal inlay as shown in FIG. 1 having a plurality of protrusions;

FIG. 8B is a cross sectional view of the corneal inlay shown in FIG. 8A along the line 8B;

FIG. 9A is a bottom view of the corneal inlay as shown in FIG. 1 having a plurality of protrusions wherein the protrusions include a partial arc;

FIG. 9B is a cross sectional view of the corneal inlay shown in FIG. 9A along the line 9B;

FIG. 10A is a bottom view of the corneal inlay as shown in FIG. 1 having a single of protrusion wherein the protrusion is in the shape of an arc;

FIG. 10B is a cross sectional view of the corneal inlay shown in FIG. 10A along the line 10B;

FIG. 11A is a bottom view of the corneal inlay as shown in FIG. 1 having a plurality of protrusions wherein the protrusions are generally straight segments;

FIG. 11B is a cross sectional view of the corneal inlay shown in FIG. 11A along the line 11B;

FIG. 12 is a bottom view of the corneal inlay according to FIG. 1 having an adhesive region according to the present invention;

FIG. 13 is a bottom view of the corneal inlay according to FIG. 1 adapted to display indicia for proper orientation;

FIG. 14 is a front plan view of the cornea having two pen dots;

FIG. 15 is a schematic representation of an aberrometer having two reticles;

FIG. 16 is a front plane view of the cornea for FIG. 14 in conjunction with the reticles of FIG. 15;

FIG. 17 is a front plane view of the cornea having a pair of dots according to one embodiment of the present invention;

FIG. 18 is a side cross sectional view of the dots shown in FIG. 17 according to one embodiment of the present invention; and

FIG. 19 is a front plane view of a cornea having the dots shown in FIG. 18 in conjunction with the reticles of FIG. 15.

### DESCRIPTION OF THE INVENTION

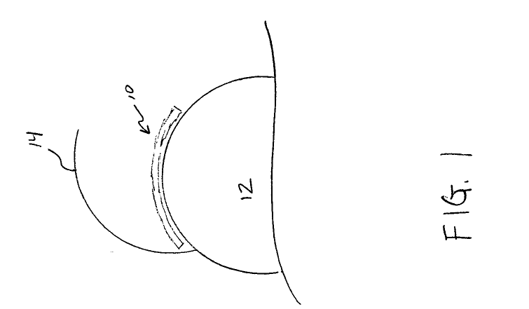

The corneal inlay 10, FIG. 1, according to the present invention, is placed on the cornea 12 under a corneal flap 14 and includes one or more of the methods according to the teachings of the present invention to prevent rotation, de-centering, and/or backwards implantation of the corneal inlay 10, as well as the precise placement of the corneal inlay 10 on the cornea 12.

Moreover, the present invention also features the use of corneal mapping techniques, such as wavefront treatment and the like, to contour the surface of the corneal inlay 10 and create customized corneal inlays 10 based on individual wavefront measurements. These customized corneal inlays 10 may be made either by the corneal inlay manufacturer or in the doctor's office. Corneal mapping techniques, and in particular wavefront correction, are capable of correcting not only lower order aberrations, but also higher order aberrations thus resulting in significantly improved vision. Using one or more of the methods and teachings described hereinbelow, the customized corneal inlay 10 may be precisely placed or aligned on the cornea 12 under the corneal flap 14, thus allowing the corneal inlay 10 to correct even minor aberrations on the cornea 12. Additionally, one or more embodiment of the present invention will prevent movement of the corneal inlay 10 once placed on the cornea 12. Should the patient's vision change, for example because of age, injury, or disease, the corneal inlay 10 is simply removed, and a new corneal inlay 10 (taking into consideration the patient's new corneal map) is created and put in its place. Because the present invention allows the corneal inlay 10 to be precisely placed on the cornea 12, the ability of a customized corneal inlay 10 to correct the patient's vision is significantly increased.

According to one embodiment, the corneal inlay 10, FIGS. 2A-2B, may include one or more protrusions 22 (for example one or more microscopic spikes 24) disposed on the bottom surface B of the corneal inlay 10. The plurality of microscopic spikes 24 engage the surface S of the cornea 12 and prevent the corneal inlay 10 from rotating and de-centering relative to the cornea 12.

As discussed above, the flexible nature of corneal inlays 10 makes determining the interior I and exterior E surfaces of the corneal inlay 10 difficult. This can lead to the corneal inlay 10 being improperly placed on the surface S of the cornea 12. The present invention solves this problem by sizing the microscopic spikes 24 such that the microscopic spikes 24 can be seen with a microscope (such as commonly used in eye surgical procedures), thereby aiding in the identification of the posterior surface of the corneal inlay 10 and reducing the likelihood that the corneal inlay 10 will be implanted inside out.

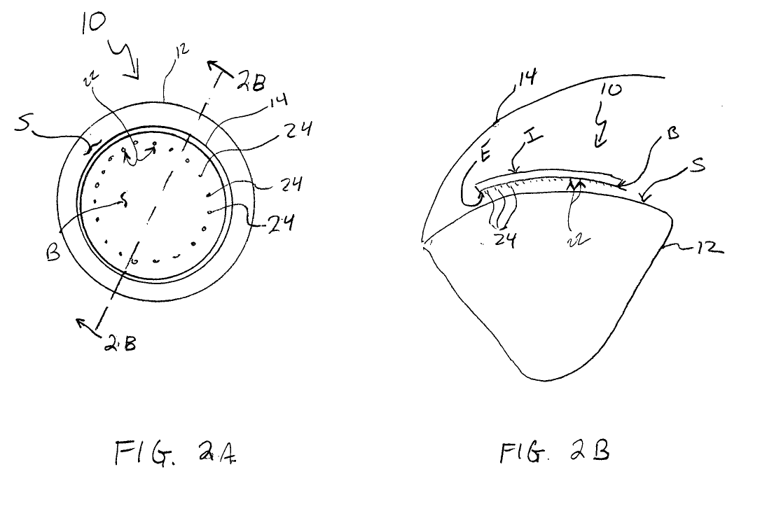

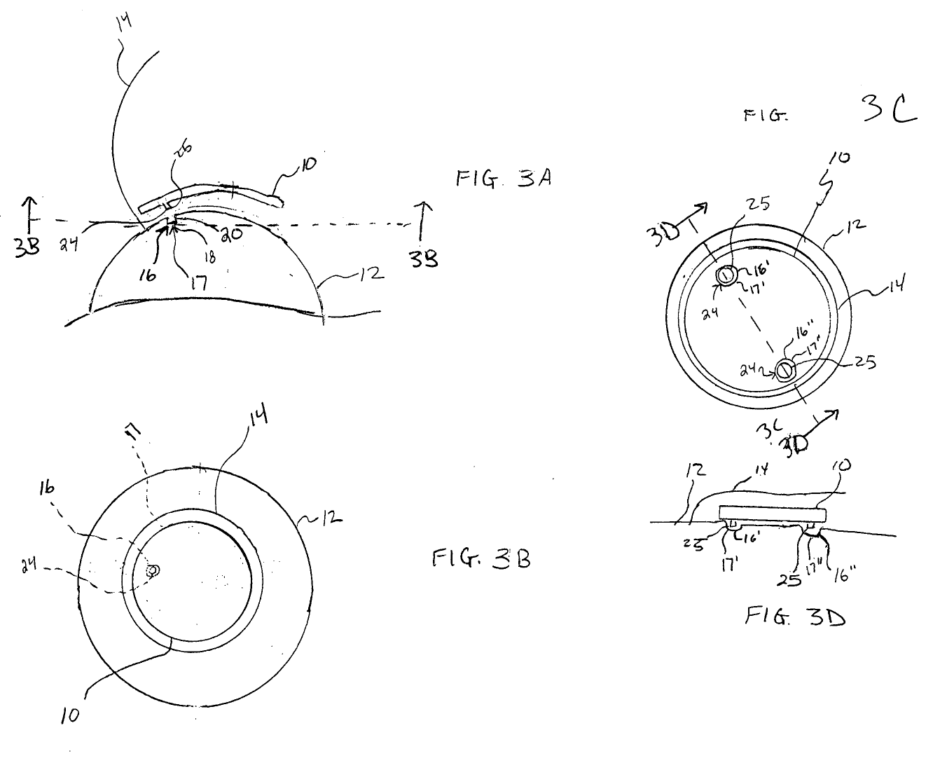

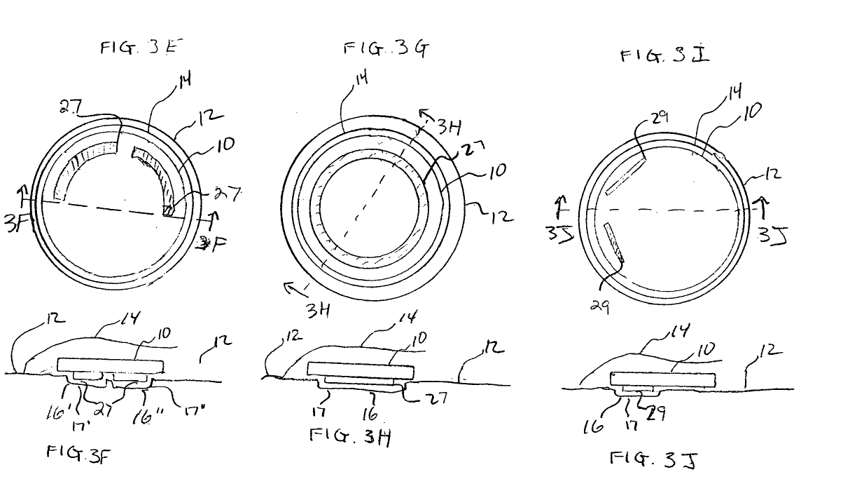

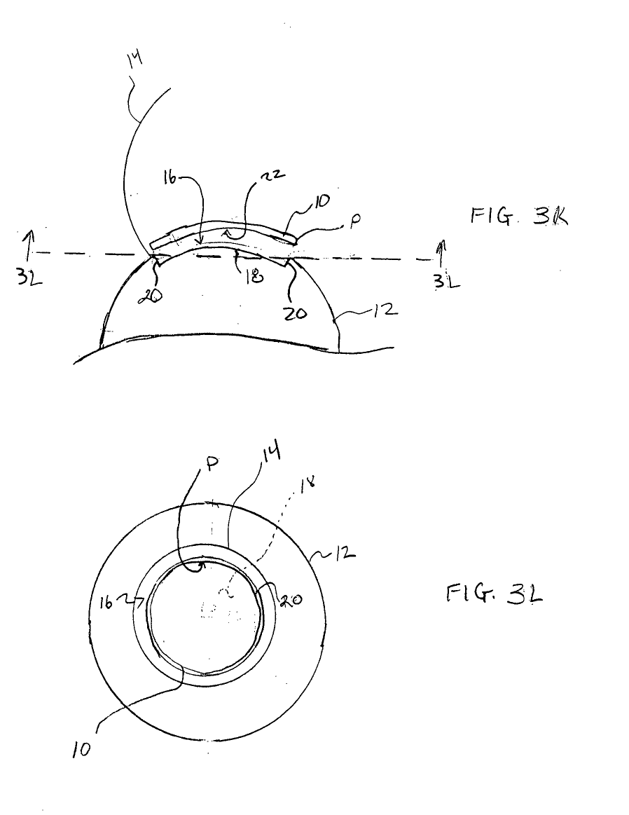

Alternatively, or in addition to, the embodiment disclosed above, the surface S of the cornea 12, FIGS. 3A-3L, may be ablated to form one or more apertures or beds 16 using any method known to those skilled in the art such as, but not limited to, laser ablation. The aperture 16 includes a base portion 18 and at least one wall portion 20. As will be discussed in greater detail hereinbelow, the aperture 16 prevents the corneal inlay 10 from moving relative to the cornea 12 by essentially providing a lock-and-key type arrangement in which the aperture 16 contains at least a portion of the corneal inlay 10.

In one embodiment, the aperture 16, FIGS. 3A-3J, may include one or more “holes” 17 that serve to receive one or more protrusions 24 on the bottom surface of the corneal inlay 10 which prevents rotation of the corneal inlay 10. The protrusions 24 also aid in identifying the posterior surface of the corneal inlay 10, thus reducing the likelihood that the corneal inlay 10 will be implanted inside out.

The protrusions 24 may include one or more posts 25, FIGS. 3A-3D having various diameters such as 0.25 to 1.0 mm, disposed about the center of the corneal inlay 10 or preferably offset from the center of the corneal inlay 10. Alternatively, the protrusions 24 may include one or more arcs 27, FIGS. 3E-3H, for example having a sweep of approximately 45-90 degrees (FIGS. 3E-3F) or approximately 360 degrees (FIGS. 3G-3H) and a depth of approximately 50-200 microns, or generally straight segments 29, FIGS. 3I-3J, for example, but not limited to, two generally straight segments disposed about 6-7 mm from the perimeter P at about 10:30and 1:30 respectively.

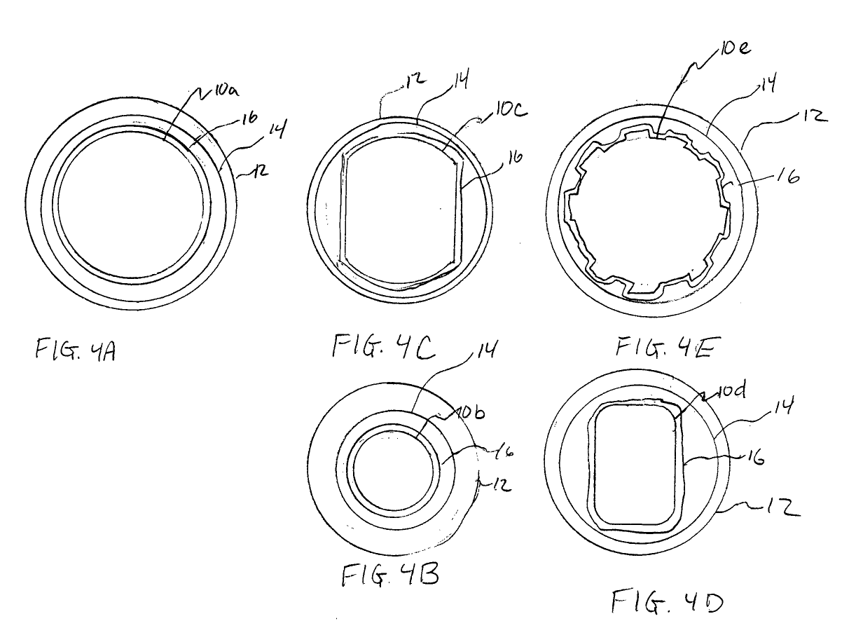

In another embodiment, the aperture 16, FIGS. 3K-3L, may be sized and shaped to contain substantially the entire corneal inlay 10. The wall portion 20 of the aperture 16 preferably generally abuts (i.e. it is in close proximity to) and follows the perimeter P of the corneal inlay 10 such that the aperture 16 and the corneal inlay 10 fit in a “lock-and-key” or “peg-in-hole” type arrangement. The aperture 16, FIGS. 4A-4E, described above may be sized and shaped to fit virtually any size or shape corneal inlay 10. For example, the corneal inlay 10 (and thus the aperture 16) may be include a generally round shape 10a, FIG. 4A, but preferably includes a non-round shape such as, but not limited to, an oval 10b, FIG. 4B, at least one side with a partial arc 10c, FIG. 4C, a rectangle 10d, FIG. 4D or a complex geometric shape having multiple facets or edges 10e, FIG. 4E for example, a circle having a pattern.

The use of a non-round corneal inlay 10, and a correspondingly non-round shaped aperture 16, prevents the corneal inlay 10 from rotating and moving about the cornea 12. Moreover, some non-round shapes (such as partial arcs and complex geometric shapes) prevent the corneal inlay 10 from accidentally being implanted inside out since they will only fit within the aperture 16 in a certain orientation. Additionally, the aperture 16 can be precisely ablated into the surface S of the cornea 12, thus allowing the corneal inlay 10 to be precisely placed on the cornea 12.

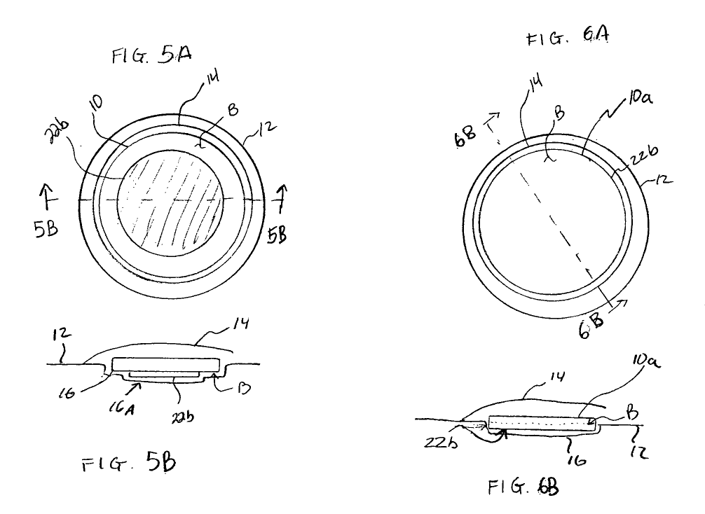

Alternatively, or in addition to, the surface of the cornea 12, FIGS. 5A-11B, preferably includes two or more of the apertures 16 described above. The cornea 12 preferably includes a first aperture 16a sized to accept the perimeter P of the corneal inlay 10 as described above. Additional apertures 16b are preferably sized and shaped to accept one or more protrusions 22 disposed on the bottom B of the corneal inlay 10 as described above. The protrusions 22 engage the apertures 16 in the “lock-and-key” or “peg-in-hole” type arrangement as discussed above, thus as preventing dislodging and/or rotation of the corneal inlay 10 relative to the cornea 12. Since the location of the apertures 16 can be precisely ablated within the cornea 12, the combination of the protrusions 22 and the apertures 16 allows for precise placement of the corneal inlay 10 on the cornea 12 as well. Moreover, the protrusion 22 aids in identifying the posterior surface of the corneal inlay 10, thus reducing the likelihood that the corneal inlay 10 will be implanted inside out.

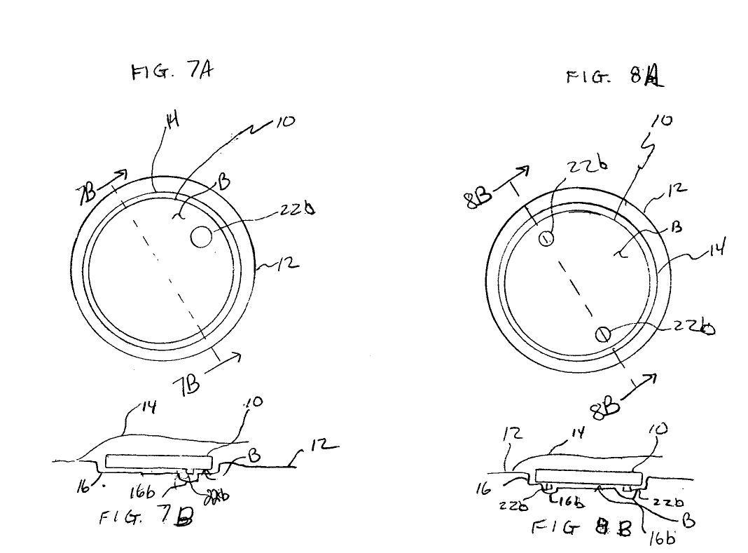

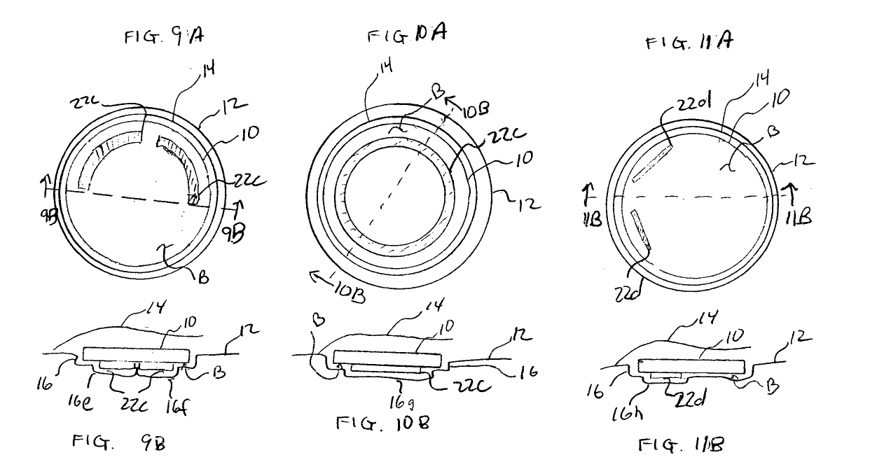

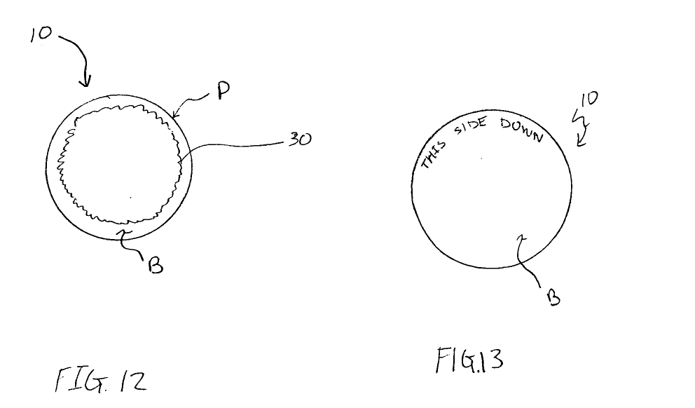

The protrusions 22 may include microscopic spikes 24 as shown in FIG. 2A-3B or one or more posts 22b, FIGS. 5-8, having various diameters from ranging from substantially the same diameter as the corneal inlay 10 (as shown in FIGS. 5 and 6) to small “pegs” (as shown in FIGS. 7 and 8) disposed about the center of the corneal inlay 10 or preferably offset from the center of the corneal inlay 10. Alternatively, the protrusions 22 may include one or more arcs 22c, FIGS. 9 and 10, for example having a sweep of approximately 45-90 degrees and a depth of approximately 50-200 microns, or generally straight segments 22d, FIG. 11, for example, but not limited to, two arcs 22d disposed about 6-7 mm from the perimeter P at about 10:30and 1:30 respectively.

According to another embodiment, the corneal inlay 10, FIG. 12, may also include an adhesive region 30 disposed about the perimeter P of the bottom surface B of the corneal inlay 10. The adhesive region 30 adheres to the surface S of the cornea 12, thus preventing any movement of the corneal inlay 10 and may include tissue glue or any other adhesive known to those skilled in the art. The corneal inlay 10, FIG. 13, may also include indicia 32 such as writing or symbols to allow for easy identification of the top and bottom of the corneal inlay 10 respectively.

When performing wavefront-based measurements with an intent to treat the wavefront abnormalities with an excimer laser (sculpting either the cornea, a corneal inlay, or a contact lens), it is imperative to register or align the wavefront profiles (wavefront error data) generated by an aberrometer with the patient's eye at the time of treatment with error data) needs to be aligned properly with respect to the cornea 12, so that the wavefront-based treatment pattern of laser shots is applied to precisely the proper areas of the cornea 12 (or corneal inlay 10 as described hereinabove). Any error in the registration, either X-Y translation, or rotationally (i.e., the eye tends to excyclotort or rotate outward slightly when the patient moves from a sitting position to a lying position) can drastically affect the results and accuracy of the custom sculpting.

In the past, the registration process involved the surgeon marking the cornea 12 or the adjacent scleral conjunctiva, FIG. 14, with two small dots 44 on either side of the cornea 12 (roughly 3 o'clock and 9 o'clock, 180 degrees apart) or on the adjacent scleral conjunctiva 40 using a surgical pen. Next, the surgeon lines up two reticles 46, FIG. 15, (which are basically small diameter, approximately 1 mm. diameter circles) on the aberrometer 48. The two reticles 46 are spaced horizontally apart (e.g., approximately 14 mm.) with a horizontal line 50 through them. The reticles 46 are adjustable such that the surgeon can move them up/down on the screen, rotate them, and move the reticles 46 in/out along the horizontal line 50 and alter the horizontal spacing. The registration process works by aligning the center of the reticles 46 with the two dots 44 marked on the cornea or scleral conjunctiva 40 as shown in FIG. 16, similar to the process of sighting a target through a scope of a gun.

This process suffers from several known problems. First, the dots 44 marked with the surgical pen MUST stay intact throughout the entire wavefront process and actual surgery. Unfortunately, these dots 44 are prone to smearing by, for example, the patient blinking or by accidental contact with the surgeon or surgical machinery. If the dots 44 are not visible when the patient is under the laser, the reticles 46 cannot be accurately aligned, and the precise registration will not be possible. Moreover, the surgical pens used in the registration process must also be thrown away afterwards because they cannot be sterilized, and the fine tip becomes blunt, thus subsequent dots have a larger diameter and become less ideal. These surgical pens are relatively expensive. Additionally, the dots 44 must be perfectly round, which can be difficult to do by hand using a pen.

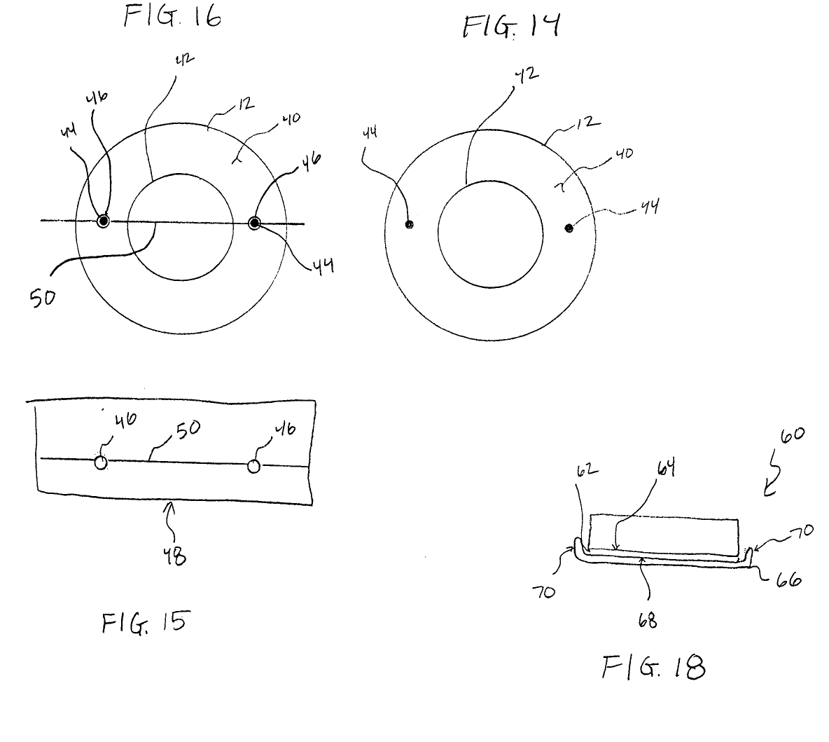

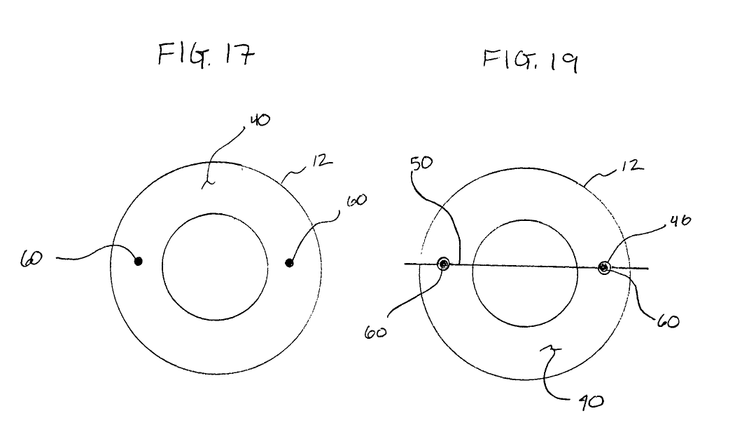

The present invention 60, FIG. 17, features a small, less than approximately 1 mm. diameter sterile paper, plastic, or the like removable segments 60. These removable segments 60 are secured to the cornea or scleral conjunctiva 40 using a surgical adhesive prior to aberrometer measurements. The removable segments 60 are preferably perfectly round, and would not move or fade or be susceptible to blinking, and would be removed after surgery. In the exemplary embodiment, the removable segments 60, FIG. 18, include an adhesive layer 62 disposed on the bottom surface 64 of the dot 60 and a release backing 66. The release backing 66 may include a thin release sheet disposed on the bottom 68 of the adhesive layer 62 of each dot 60 and preferably includes one or more tabs 70 to aid in removing the release backing 66 from the adhesive layer 62. Alternatively, the release backing 66 may includes a larger release sheet having two or more removable segments 60. In another embodiment, the surgeon may place a small area of surgical adhesive directly on the cornea or scleral conjunctiva 40 and then place the removable segments 60 in the adhesive.

The removable segments 60 preferably have a color that contrasts with the cornea or sclera 40, for example, without limitation, black or the like. In the exemplary embodiment, the removable segments 60 have a perfectly round shape, though other shapes such as, but not limited to, straight lines or the like, are also envisioned. The dots are preferably constructed from a material that is safe and non-toxic to the eye.

The removable segments 60, FIG. 19, could be installed by grabbing them with sterile forceps, peeling the release backing 66 (i.e., when the removable segments 60 include an integral adhesive layer 62) or applying an adhesive dot to the cornea 12 (i.e., when the removable segments 60 do not include an integral adhesive layer 62), and placing them on the (anesthetized) conjunctiva overlying the sclera 40, near the limbus. The removable segments 60 could then be easily removed using the sterile forceps immediately following the surgical procedure.

Modifications and substitutions by one ordinary skill in the art are considered to be within the scope of the present invention.

The invention claimed is:

1. A corneal inlay comprising a top and at least a bottom surface, wherein said bottom surface includes at least one protrusion extending downwardly, wherein said protrusion is adapted to engage a surface of a cornea.

2. The corneal inlay as claimed in claim 1 wherein said at least one protrusion includes a plurality of microscopic spike like protrusions.

3. The corneal inlay as claimed in claim 1 wherein said at least one protrusion is disposed offset from a center of said corneal inlay.

4. The cornea inlay as claimed in claim 1 wherein said at least one protrusion includes a generally cylindrical protrusion.

5. The corneal inlay as claimed in claim 1 wherein said at least one protrusion includes a generally arc shaped protrusion.

6. The corneal inlay as claimed in claim 1 wherein said at least one protrusion includes a generally ring shaped protrusion, wherein said protrusion is disposed proximate a perimeter of said bottom surface of said corneal inlay.

7. The corneal inlay as claimed in claim 1 wherein said at least one protrusion includes a generally straight segment.

8. The corneal inlay as claimed in claim 1 wherein said at least one protrusion includes a single protrusion having substantially the same dimensions as said bottom surface of said corneal inlay.

9. The corneal inlay as claimed in claim 1 wherein said at least one protrusion is sized and shaped to fit within at least one aperture disposed in said cornea.

10. The corneal inlay as claimed in claim 9 wherein said at least one protrusion includes a non-circular outer perimeter corresponding to a non-circular outer perimeter of said at least one aperture.

11. The corneal inlay as claimed in claim 9 wherein said at least one protrusion and said at least one aperture are disposed offset from a center of said corneal inlay in said cornea respectively.

12. The cornea inlay as claimed in claim 9 wherein said at least one protrusion includes a generally cylindrical protrusion sized and shaped to fit within a generally cylindrical aperture.

13. The corneal inlay as claimed in claim 9 wherein said at least one protrusion includes a generally arc shaped protrusion sized and shaped to fit within a generally arc shaped aperture.

14. The corneal inlay as claimed in claim 9 wherein said at least one protrusion includes a generally ring shaped protrusion sized and shaped to fit within any generally ring shaped aperture, wherein said protrusion and said aperture are disposed proximate a perimeter of said bottom surface of said corneal inlay.

15. The corneal inlay as claimed in claim 9 wherein said at least one protrusion includes a generally straight segment sized and shaped to fit within a generally straight aperture.

16. The corneal inlay as claimed in claim 9 wherein said at least one protrusion includes at least two protrusions disposed about said bottom surface of said corneal inlay and sized and shaped to get within at least two apertures disposed in said cornea.

17. A corneal inlay comprising a top surface, a bottom surface, and a non-circular perimeter, wherein said non-circular perimeter of said corneal inlay is sized and shaped to fit within an aperture having a non-circular outer perimeter disposed in a cornea.

18. The corneal inlay as claimed in claim 17 wherein said non-circular perimeter of said corneal inlay includes a generally oval shape.

19. The corneal inlay as claimed in claim 17 wherein said corneal inlay includes at least one protrusion disposed about said bottom surface, wherein said at least one protrusion includes a non-circular perimeter.

20. A corneal inlay comprising a top surface, a bottom surface, and an adhesive layer disposed about at least a portion of said bottom surface of said corneal inlay, wherein said adhesive layer adheres said corneal inlay to a surface of a cornea.

21. The corneal inlay as claimed in claim 20 wherein said adhesive is disposed proximate an outer region of said bottom surface of said corneal inlay.

22. The corneal inlay as claimed in claim 20 wherein said corneal inlay further includes release backing disposed about a lower surface of said adhesive layer.

23. The corneal inlay as claimed in claim 20 wherein said corneal inlay further includes indicia disposed proximate an outer region of said corneal inlay, wherein said indicia is indicative of a proper orientation of said corneal inlay.

24. A method of preventing movement of a corneal inlay relative to a surface of a cornea upon which said corneal inlay is disposed, said method comprising the acts of:
placing a corneal inlay having at least one protrusion disposed about and extending downwardly from a bottom surface of said corneal inlay on said surface of said cornea; and
engaging said surface of said cornea with said least one protrusion.

25. The method as claimed in claim 24 wherein said at least one protrusion of said corneal inlay includes a plurality of microscopic spike like protrusions.

26. The method as claimed in claim 24 further including the act of cutting or ablating said surface of said cornea to form a corneal flap, wherein said corneal inlay is disposed beneath said corneal flap on said surface of said cornea.

27. The method as claimed in claim 24 further including the act of ablating said surface of said cornea to form at least one aperture, wherein said aperture is sized and shaped to accept said at least one protrusion such that said aperture in said protrusion form a lock and key type arrangement.

28. The method as claimed in claim 27 wherein said at least one protrusion is disposed about an outer region of said bottom surface of said corneal inlay, wherein said act of ablating said surface of said cornea to form said at least one aperture includes forming said aperture about an outer region of said cornea where said corneal flap is to be disposed.

29. A method of preventing movement of a corneal inlay relative to a surface of a cornea upon which said corneal inlay is disposed, said method comprising the acts of:
ablating said surface of said cornea to form at least one aperture having a non-circular outer perimeter corresponding to a non-circular perimeter of a corneal inlay; and
placing at least a portion of said non-circular perimeter of said corneal inlay within said non-circular aperture, wherein said non-circular corneal inlay and said non-circular aperture form a lock and key type arrangement.

30. The method as claimed in claim 29 wherein an outer perimeter of said corneal inlay forms said non-circular perimeter of said corneal inlay.

31. The method as claimed in claim 29 wherein said act of ablating said surface of said cornea to form said at least one aperture includes forming at least one non-circular aperture size and shaped to accept at least one non-circular protrusion disposed about a bottom surface of said corneal inlay.

32. The method as claimed in claim 31 wherein said act of ablating said at least one non-circular aperture includes forming an aperture having a generally arc shape corresponding to a generally arc shape protrusion of said corneal inlay.

33. The method as claimed in claim 31 wherein said act of ablating said at least one non-circular aperture includes forming an aperture having a generally rectangular shape corresponding to a generally rectangular shape protrusion of said corneal inlay.

34. The method as claimed in claim 31 further including the act of identifying a proper orientation of said corneal inlay by using said at least one protrusion.

35. The method as claimed in claim 29 further including the act identifying a proper orientation of said corneal inlay by viewing indicia disposed on at least a portion of said corneal inlay.

36. The method as claimed in claim 29 further including the act of securing said corneal inlay to said cornea using an adhesive.

37. A method of orienting a corneal inlay relative to a surface of a cornea comprising the acts of:
forming a corneal inlay having at least one protrusion extending downwardly from a bottom surface of said corneal inlay;
ablating said surface of said cornea to form at least one aperture size and shaped to accept said protrusion; and
inserting said protrusion within said aperture.

38. The method as claimed in claim 37 wherein said at least one protrusion is disposed about an outer region of said corneal inlay.

39. A method of performing wavefront-based measurements comprising the acts of:
securing at least one removable segment to a surface of a cornea or scleral conjunctiva;
aligning an aberrometer with a patient's cornea using said at least one removable segment; and
generating wavefront error data.

40. The method as claimed in claim 39 wherein said act of securing said at least one removable segment to said surface of said cornea or scleral conjunctiva includes securing said at least one movable segment using an adhesive.

41. The method as claimed in claim 39 further including the act of removing a release backing from a lower surface of an adhesive layer disposed on a bottom surface of said removable segment thereby exposing said adhesive layer.

42. The method as claimed in claim 39 wherein said removable segment includes a color which is in contrast to a color of said cornea or sclera.

43. The method as claimed in claim 39 wherein said movable segment is formed from plastic.

44. The method as claimed in claim 39 wherein said removable segment is formed from paper.

45. The method as claimed in claim 39 wherein said movable segment is secured to the cornea or scleral conjunctiva.
